(19) United States
(12) Patent Application Publication
(10) Pub. No.: US 2016/0089489 A1
(43) Pub. Date: Mar. 31, 2016

(54) MEDICAL IMAGING FACILITY AND METHOD FOR CONTROL OF A CONTRAST AGENT INJECTOR INTEGRATED WITHIN A MEDICAL IMAGING FACILITY

(71) Applicant: Siemens Aktiengesellschaft, Muenchen (DE)

(72) Inventor: Martin Ringholz, Erlangen (DE)

(73) Assignee: SIEMENS AKTIENGESELLSCHAFT, Muenchen (DE)

(21) Appl. No.: 14/865,236

(22) Filed: Sep. 25, 2015

(30) Foreign Application Priority Data
Sep. 26, 2014 (DE) 102014219529.2

(51) Int. Cl. A61M 5/00; A61B 6/03

(52) U.S. Cl. CPC A61M 5/007; A61B 6/037; A61M 2205/50

(57) ABSTRACT

A medical imaging facility that has a signal detection unit with a patient accommodation area that accommodates a patient for a medical imaging investigation. The patient accommodation area is at least partially surrounded by the signal detection unit, and the medical imaging facility has a contrast agent injector that is integrated within the medical imaging facility.

### BACKGROUND OF THE INVENTION

1. Field of the Invention

The present invention concerns a medical imaging facility with a contrast agent injector. The present invention also concerns a method for control of a contrast agent injector integrated within a medical imaging facility, and a non-transitory, computer-readable storage medium encoded with programming instructions for implementing such a method.

2. Description of the Prior Art

Continually, a contrast agent injector has been used for medical imaging investigations of a patient that necessitate an injection of a contrast agent during the medical imaging investigation. Conventional contrast agent injectors are designed as units that are separate from the medical imaging facility with which the medical imaging investigation is carried out.

### SUMMARY OF THE INVENTION

An objective of the present invention is to provide a simple way in which a contrast agent injector can be controlled by the medical imaging facility during a medical imaging investigation.

This object is achieved in accordance with the invention by a medical imaging facility with a signal detection unit having a patient receptacle designed to accommodate a patient for a medical imaging investigation, wherein the patient receptacle is at least partially surrounded by the signal detection unit.

The medical imaging facility has a contrast agent injector that is integrated within (built into) the medical imaging facility. In this context, the term “signal detection unit” is to be understood in particular as the scanner of the medical imaging facility. If the medical imaging facility is in the form, for example, of a magnetic resonance facility, the signal detection unit incorporates a magnetic unit with a superconducting basic field magnet, a gradient coil unit and a radio-frequency antenna unit. The term “contrast agent injector” means a unit that incorporates at least one container for a contrast agent. The contrast agent can be administered to the patient by injection by the contrast agent injector. The contrast agent injector that is integrated within the medical imaging facility is preferably incorporated into the medical imaging facility, so that the control of the contrast agent injector is effected solely by a control computer of the medical imaging facility. In this case, the contrast agent injector cannot be used and/or operated independently of the medical imaging facility, i.e., it is not a “stand alone” unit.

The invention provides a simple way of controlling a contrast agent injector via the medical imaging facility during a medical imaging investigation, because the contrast agent injector is already integrated within the medical imaging facility and can be directly controlled by a system control computer of the medical imaging facility. In addition, the workflow for the preparation of a medical imaging investigation can be simplified for medical operating staff, because the coupling of a contrast agent injector onto the medical imaging facility and/or onto a patient positioning device, as is conventional, is no longer necessary.

The medical imaging facility has a housing that screens (shields) the signal detection unit and which at least partially surrounds the patient accommodation area, and the housing has a contrast agent storage unit for the storage and/or arrangement of the contrast agent injector. In this way, a particularly simple arrangement of the contrast agent injector within the medical imaging facility can be achieved. In addition, with the contrast agent storage unit, the contrast agent injector can be protected from unwanted damage. Further, it is possible to achieve particularly simple accessibility to the contrast agent injector for the medical operating staff, for example to replenish and/or exchange a contrast agent.

It is particularly advantageous for the housing to have a housing shell on which the contrast agent storage unit is arranged, so that it is possible to achieve a retrofitted arrangement and/or mounting of the contrast agent storage unit on the medical imaging facility, in particular on a medical imaging facility that is already in clinical use.

As an alternative, the housing shell can have a recess in which the contrast agent storage unit is arranged, at least partially. This achieves an especially space saving arrangement and/or integration of the contrast agent injector within the medical imaging facility.

An especially advantageous accessibility of the contrast agent injector for medical operating staff is achieved in an embodiment wherein the contrast agent storage unit is arranged outside the patient accommodation area. In addition, this embodiment prevents any undesired influence on the medical imaging investigation and/or undesired hindrance to the patient during the medical imaging investigation, due to the arrangement of the contrast agent unit outside the patient accommodation area.

In a further embodiment of the invention, the contrast agent storage unit is arranged on a front side of the housing. This embodiment also provides accessibility to the contrast agent injector for medical operating staff. This achieves a simple patient preparation, for example when bringing injection components of the contrast agent injector up to the patient, because the contrast agent injector is situated immediately in an accommodation area for the patient into whom the components are inserted immediately before the medical imaging investigation.

In a further embodiment, the medical imaging facility has a contrast agent control computer that is designed for control of the contrast agent injector, with the contrast agent control computer being linked with the contrast agent injector for data exchange. This allows a simple and particularly time-saving control of the contrast agent injector by the medical imaging facility during a medical imaging investigation. This facilitates a method for controlling a contrast agent injector that which is integrated within a medical imaging facility.

In this embodiment, the link between the contrast agent control computer and the contrast agent injector, for the purpose of data exchange, can be wireless (cable-free). The data exchange can include both the transmission of control signals from the contrast agent control computer to the contrast agent injector and the transmission of data signals from the contrast agent injector to the contrast agent control computer. The contrast agent control computer can in addition be integrated within a system control computer of the medical imaging facility. Alternatively, the contrast agent control computer can be a separate item from the system control computer of the medical imaging facility.

The invention also encompasses a method for controlling a contrast agent injector that is integrated within a medical imaging facility, that includes the following steps of determining at least one contrast agent parameter of the contrast agent injector by operation of the contrast agent control computer, generating at least one control parameter for control of the contrast agent injector using the at least one contrast agent parameter, and controlling the contrast agent injector with the at least one control parameter.

A simple and time-saving control of the contrast agent injector is achieved by the medical imaging facility during a medical imaging investigation. In addition, the workflow for the preparation for a medical imaging investigation is simplified, because it is no longer necessary to couple the contrast agent injector to the medical imaging facility for the medical imaging investigation.

Preferably, the medical imaging facility will in this embodiment incorporates a signal detection unit, having a patient accommodation area that is designed to accommodate a patient for a medical imaging investigation, with the patient accommodation area being at least partially surrounded by the signal detection unit. In addition, the medical imaging facility incorporates a system control computer, the contrast agent injector and the contrast agent control computer. The contrast agent control computer can be integrated within a system control computer of the medical imaging facility. Alternatively, the contrast agent control computer can be separate from the system control computer of the medical imaging facility. The determination of the at least one contrast agent parameter can also include data transmission of signal data from the contrast agent injector to the contrast agent control computer. Further, the control of the contrast agent injector can include the transmission of control data and/or control parameters from the contrast agent control computer to the contrast agent injector.

The advantages of the inventive method for controlling a contrast agent injector that is integrated within a medical imaging facility correspond essentially to the advantages of the inventive medical imaging facility, which are described above. Characteristics, advantages or alternative forms of embodiment described above apply to the other aspects of the invention.

In a further embodiment, before the generation of the at least one control parameter by the contrast agent control computer, an investigation parameter for the impending imaging investigation is detected, and the control parameter for the control of the contrast agent injector is generated by the contrast agent control computer dependent on the investigation parameter. Within the contrast agent control computer, both data about the contrast agent injector, for the control of the contrast agent injector, and relevant investigation data and/or investigation parameters for the medical imaging investigation, can be brought together and the control parameters for the contrast agent injector can be determined therefrom by the contrast agent control computer. The control parameter for controlling the contrast agent injector is thus adapted both to individual contrast agent parameters of the contrast agent injector and to individual investigation parameters of the selected medical imaging investigation. The investigation parameter can be, for example, a sequence parameter for a magnetic resonance investigation and/or another parameter that appears reasonable to a specialist. In addition, the investigation parameter can include information as to whether a contrast agent injection is necessary for the impending medical imaging investigation, and insofar as necessary, the investigation parameter can also contain information as to which contrast agent is required.

In a further embodiment of the invention, the at least one contrast agent parameter includes a level parameter for at least one contrast agent in the contrast agent injector and/or a substance parameter for the at least one contrast agent in the contrast agent injector. By reference to the contrast agent parameter, the contrast agent control computer can thus check and/or monitor whether the contrast agent required for a desired medical imaging investigation is present in a sufficient quantity within the contrast agent injector. It is also possible by reference to the level parameter, and hence by reference to an available quantity of contrast agent for at least one contrast agent in the contrast agent injector, for the contrast agent control computer to calculate an expected number of applications which are still possible, and to communicate this to medical operating staff, for example via a user interface of the medical imaging facility.

The method for control of the contrast agent injector that is arranged within the medical imaging facility, can include the following steps. The contrast agent control computer can determine at least one parameter related to an expected complete exhaustion of the at least one contrast agent, that is still present, by reference to the at least one contrast agent parameter, and the at least one parameter related to the expected complete exhaustion of the at least one contrast agent can be presented at a presentation unit.

In this context, a parameter of an expected exhaustion of the at least one contrast agent which is still present means an expected number of applications that are still possible using the at least one contrast agent that is still present, within the contrast agent control injector. Furthermore, the term “presentation unit” means a unit of the medical imaging facility that is designed for the storage and/or output and/or display of the at least one parameter for the expected complete exhaustion of the at least one contrast agent. Here, the presentation unit can incorporate a storage unit and/or an output unit, for example a monitor, etc.

This embodiment of the invention enables medical operating staff to be informed in good time about an expected complete exhaustion of the at least one contrast agent, who can then advantageously undertake any necessary replenishment of the at least one contrast agent and/or the ordering of the at least one contrast agent.

In a further embodiment, if the at least one parameter for the expected complete exhaustion of the at least one contrast agent falls below a threshold value, the contrast agent control computer will generate a purchase order for a new quantity of the at least one contrast agent. This ensures that an adequate quantity of the at least one contrast agent is always available in stock. The purchase order is preferably produced by the contrast agent control computer. Dispatch of the purchase order that has been produced can also be effected by the contrast agent control computer, for example by suitable software therein. It is also possible for the purchase order that has been produced to be dispatched manually by the medical operating staff.

In a further embodiment of the invention, the method for controlling the contrast agent injector, that is arranged within a medical imaging facility, includes the following steps. A warning message is generated by the contrast agent control computer in the event of a detected absence of the at least one contrast agent and/or the detected presence of a quantity of the at least one contrast agent that is below a threshold value, and the warning message is presented by the presentation unit.

In this embodiment, therefore, before a medical imaging investigation, it is possible to call the attention of medical operating staff to the fact that a planned medical imaging investigation is not possible with the at least one contrast agent, or that the at least one contrast agent must first be replenished with a new quantity. The warning message is shown at a display unit of the medical imaging facility, such as a monitor.

The present invention also encompasses a non-transitory, computer-readable data storage medium that is loadable into a memory of a programmable contrast agent control computer of a medical imaging facility, with a contrast agent injector built into the medical imaging facility. The storage medium is encoded with programming instructions that cause the contrast agent control computer to implement any or all embodiments of the inventive method as described above, when the programming instructions are executed with contrast agent control computer.

### BRIEF DESCRIPTION OF THE DRAWINGS

FIG. 1 schematically illustrates a medical imaging facility in accordance with the invention.

FIG. 2 schematically illustrates a first exemplary embodiment of the inventive medical imaging facility.

FIG. 3 schematically illustrates a second exemplary embodiment of the inventive medical imaging facility.

FIG. 4 is a flowchart for the method in accordance with the invention for controlling a contrast agent injector that is integrated within a medical imaging facility.

### DESCRIPTION OF THE PREFERRED EMBODIMENTS

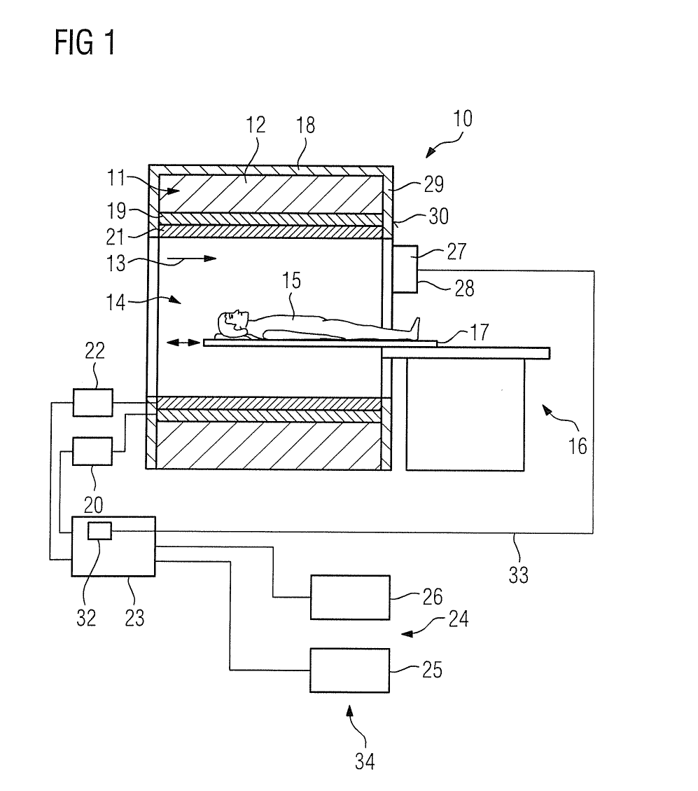

FIG. 1 schematically shows a medical imaging facility. In the present exemplary embodiment, the medical imaging facility is in the form of a magnetic resonance facility 10. However, the form of the medical image facility is not restricted to a magnetic resonance facility 10. Rather, the medical imaging facility can be in the form of any medical imaging facility, such as a computed tomography facility, a positron-emission-tomography facility, a C-arm facility, etc.

The magnetic resonance facility 10 has a signal detection unit 11, which is formed by a scanner in the magnetic resonance facility 10. The scanner has a superconducting basic field magnet 12 for the production of a strong and constant basic magnetic field 13. The magnetic resonance facility 10 has a patient accommodation area 14 for accommodating a patient 15. In the exemplary embodiment, the patient accommodation area 14 is cylindrical in shape and in a circumferential direction is cylindrically surrounded by the scanner. In principle, however, a different construction of the patient accommodation area 14 is possible. Using a patient positioning device 16 of the magnetic resonance facility 10, the patient 15 can be moved into the patient accommodation area 14. For this purpose, the patient positioning device 16 has a patient table 17 designed to be movable within the patient accommodation area 14. The magnetic resonance facility 10 has a housing 18 that screens (shields) the signal detection unit 11, from outside, and in the present exemplary embodiment surrounds the patient accommodation area 14 cylindrically.

The scanner has a gradient coil unit 19 for the generation of magnetic field gradients, which are used for spatially encoding the detected magnetic resonance signals during the imaging. The gradient coil unit 19 is controlled by a gradient controller 20 in the magnetic resonance facility 10. The scanner also has a radio-frequency antenna unit 21 for exciting nuclear spins in the patient so as to deviate from the excitation of a polarization established in the basic magnetic field 13 produced by the basic magnet 12. The radio-frequency antenna unit 21 is controlled by a radio-frequency antenna controller 22 of the magnetic resonance facility 10, so as to radiate radio-frequency magnetic resonance sequences into an examination space, which is essentially formed by the patient accommodation area 14 of the magnetic resonance facility 10.

For the controlling the basic field magnet 12, the gradient controller 20 and the radio-frequency antenna controller 22, the magnetic resonance facility 10 has a system controller 23. The system controller 23 exercises central control over the magnetic resonance facility 20, such as for the execution of a predetermined imaging gradient echo sequence. In addition, the system controller 23 incorporates an evaluation unit, not shown in more detail, for the evaluation of items of medical image data which are detected during the magnetic resonance investigation. In addition, the magnetic resonance facility 10 has a user interface 24, which is linked to the system controller 23. The system controller 23 and all of the aforementioned controllers, are formed as one or more computers. Control data, such as for example imaging parameters, together with reconstructed magnetic resonance images, can be displayed for medical operating staff on a display unit 25 of the user interface 24, for example on at least one monitor. Further, the user interface 24 has an input unit 26, via which data and/or parameters can be input by the medical operating staff during a measurement procedure.

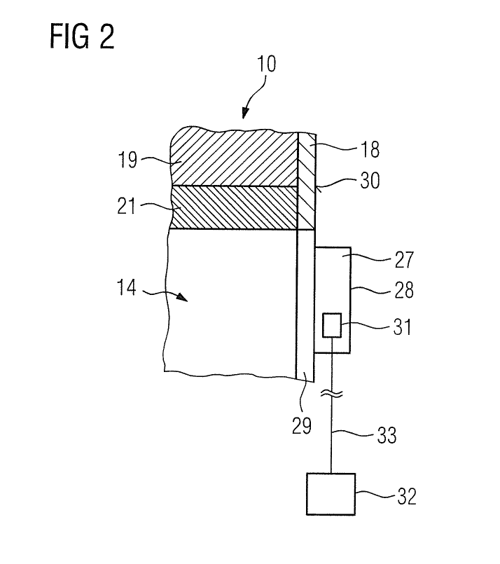

The magnetic resonance facility 10 also has a contrast agent injector 27, which is integrated within the magnetic resonance facility 10 (FIGS. 1 and 2). For this purpose, the housing 18 of the magnetic resonance facility 10 has a contrast agent storage unit 28, which is designed for storing and/or receiving the contrast agent injector 27. In the following, examples of the contrast agent injector 27 and the contrast agent storage unit 32 will be described in more detail with reference to the exemplary embodiment in FIG. 2.

The contrast agent storage unit 28 is arranged on a housing shell 29 of the housing 18. In the shown embodiments, the contrast agent storage unit 28 is arranged on a side of the housing shell 29 facing away from the scanner. For advantageous accessibility of the contrast agent unit 27 by medical operating staff, the contrast agent storage unit 28 is arranged outside the patient accommodation area 14. In particular, in this case the contrast agent storage unit 28 is arranged on a front side 30 of the housing 18. The term “front side” 30 of the housing 18 is to be understood as a side of the housing that has an infeed opening into the patient accommodation area 14, for introducing the patient 15 into the patient accommodation area 14 (FIG. 2).

The contrast agent storage unit 28 incorporates individual housing elements, which surround the contrast agent injector 27. For example, the contrast agent storage unit 28 can incorporate a cabinet and/or box, within which is arranged the contrast agent injector 27.

The contrast agent injector 27 has several contrast agent containers, not shown in more detail, for the accommodation of contrast agents and/or of salt solutions. In the present exemplary embodiment, the contrast agent unit 27 has at least two or more contrast agent containers, while a different construction of the contrast agent unit 27 is always possible. In addition, the contrast agent injector 27 includes an injection stage 31 for the actual interaction with the patient to administer the contrast agent and/or saline solutions to a patient 15 (FIG. 2).

For controlling the contrast agent injector 27, the medical imaging facility 10 has a contrast agent control computer 32 (FIGS. 1 and 2). In the exemplary embodiment, the contrast agent control computer 32 is incorporated into the system controller 23. In principle, however, it is for the contrast agent control computer 32 to be a separate item from the system controller 23. For controlling the contrast agent injector 27, the contrast agent control computer 32 has software and/or computer programs as required for this purpose, these being stored in a memory of the contrast agent control computer 32 (not shown). The programs and/or software have program capabilities designed for controlling the contrast agent injector 27 when the programs and/or software are executed by a processor in the contrast agent control computer 32.

For the purpose of data exchange, the contrast agent control computer 32 is linked to the contrast agent unit 27 by a data transmission unit 33. The data transmission unit 33 can enable cable-based data transmission between the contrast agent control unit 32 and the contrast agent injector 27. Cable-free (wireless) data transmission between the contrast agent control unit 32 and the contrast agent unit 27 is also possible.

Furthermore, the magnetic resonance facility 10 has a presentation unit 34. Here, the presentation unit 34 can incorporate the user interface 24 and/or a memory, for example a memory integrated within the contrast agent control computer 32.

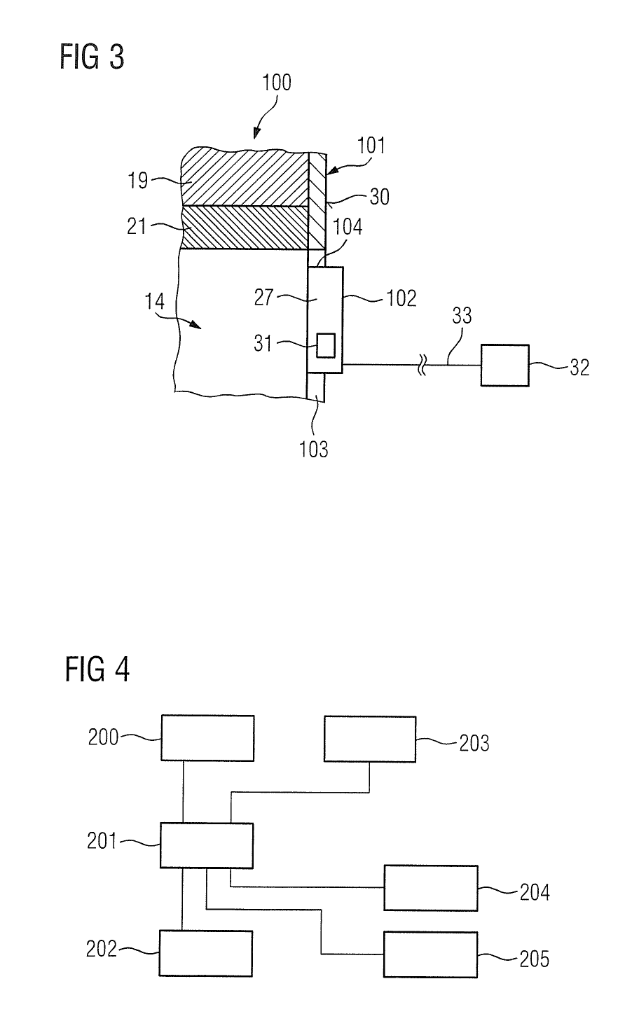

FIG. 3 shows an alternative exemplary embodiment of the magnetic resonance facility 100. Components, features and functions which remain essentially the same have been labeled with the same reference marks. The description which follows is restricted to the differences from the exemplary embodiment in FIGS. 1 and 2. Reference should be made to the description of the exemplary embodiment in FIGS. 1 and 2 for components, features and functions that remain the same.

The magnetic resonance facility 100 in FIG. 3 differs from the magnetic resonance facility 10 in FIG. 2 with respect to the form of the housing 101 and the arrangement of a contrast agent storage unit 102 on the housing 101. The housing 101 of the magnetic resonance facility 100 in FIG. 3 has a housing shell 103 with a recess 104. The contrast agent storage unit 102 of the magnetic resonance facility 100 is arranged within the recess 104, thus making possible a particularly space-saving integration of the contrast agent storage unit 102 within the magnetic resonance facility 100, in particular within the housing 101.

Analogous to the exemplary embodiment in FIG. 2, the contrast agent storage unit 102 is arranged outside a patient accommodation area 14 of the magnetic resonance facility 100. The contrast agent storage unit 102 is also arranged on a front side 30 of the housing unit 101.

The further form of the contrast agent storage unit 102, and the contrast agent injector 27 arranged within the contrast agent storage unit 102, correspond to the embodiments of the exemplary embodiment in FIGS. 1 and 2. In addition, the further form of the magnetic resonance facility 100 corresponds to the embodiments of the exemplary embodiment in FIGS. 1 and 2.

FIG. 4 shows in more detail the execution sequence for a method in accordance with the invention, for controlling the contrast agent injector 27 that is integrated within the magnetic resonance facility 10, 100. The method can be executed automatically by the contrast agent control computer 32, before a medical imaging investigation on the patient 15. In addition, the method can be started manually by medical operating staff before the medical imaging investigation. Alternatively or additionally, a manual start for the method by the medical operating staff is also conceivable if they wish to check the contrast agent injector 27, in particular as to the type and/or quantity of the individual contrast agents and/or saline solutions.

Here, in a first step 200 of the method the contrast agent control unit 32 determines at least one contrast agent parameter of the contrast agent injector 27. The determination of the at least one contrast agent parameter can here include the detection of items of contrast agent data for the contrast agent injector 27, these being transmitted directly from the contrast agent injector 27 to the contrast agent control computer 32 by means of the data transmission unit 33. Using the items of contrast agent data which has been transmitted, at least one contrast agent parameter is thereby detected and/or determined within the contrast agent control computer 32.

The at least one contrast agent parameter which is determined by the contrast agent control computer 32 can here include a level parameter for at least one of the contrast agents which are present within the contrast agent injector 27 and/or for a saline solution which is present within the contrast agent injector 27. The level parameter can indicate a remaining quantity of contrast agent and/or saline solution that is currently present within the contrast agent injector 27. Alternatively or in addition, the at least one contrast agent parameter that is determined by the contrast agent control computer 32 can include a substance parameter for at least one of the contrast agents which is present within the contrast agent injector 27 and/or for a saline solution that is present within the contrast agent injector 27. The substance parameter can indicate the nature and/or a characteristic of the substance for the contrast agent and/or saline solution that is present within the contrast agent injector 27.

In a further step 201 of the method, the contrast agent control computer 32 generates, dependent on the at least one contrast agent parameter, at least one control parameter for the control of the contrast agent injector 27. In addition, depending on the at least one contrast agent parameter and/or the at least one control parameter, a user message can be generated in the further step 200 of the method, and this user message can be communicated directly to the medical operating staff via the presentation unit 34, in particular the user interface 24, in a further step 205 of the method. The user message can, for example, include a note that a desired contrast agent injection can be started using the contrast agent injector 27. Furthermore, the user message can also include a warning message, which can be generated by the contrast agent control computer 32 in the step 201 of the method. The contrast agent control computer 32 can here generate a warning message if, for example, the contrast agent which is stored and/or available within the contrast agent injector 27 is not the correct and/or desired contrast agent. Further, the contrast agent control computer 32 can here generate a warning message if, for example, an inadequate quantity of a contrast agent is available within the contrast agent injector 27 for the impending medical imaging investigation.

Following this, in a further step 202 of the method, the contrast agent injector 27 is controlled by the contrast agent control computer 32, by the at least one control parameter. If, for example, in the case of an impending magnetic resonance investigation that calls for the injection of a contrast agent, the contrast agent required for the magnetic resonance investigation is present in sufficient quantity within the contrast agent injector 27, the control parameter is designed such that an injection of the contrast agent by the contrast agent injector 27 can be started by the contrast agent control computer 32 and the at least one control parameter. If, however, in the case of an impending magnetic resonance investigation that calls for the injection of a contrast agent, the contrast agent required for the magnetic resonance investigation is not present in sufficient quantity within the contrast agent injector 27 or is not available at all within the contrast agent unit 27, the control parameter is designed such that an injection of the contrast agent by the contrast agent injector 27 is blocked by the contrast agent control computer 32 and the at least one control parameter. Transmission of the at least one control parameter from the contrast agent control computer 32 to the contrast agent injector 27 is effected by the data transmission unit 33.

In a further step 203 of the method, an investigation parameter for an impending medical imaging investigation, in particular a magnetic resonance investigation, can be detected by the contrast agent control computer 32. The detection of an investigation parameter can include, for example, the communication to the contrast agent control computer 32 of a sequence parameter for a selected magnetic resonance sequence and/or selection of contrast agents required and/or relevant for the medical imaging investigation. By reference to the investigation parameter, it is possible in step 201 of the method, in the generation of the at least one control parameter for controlling the contrast agent injector 27, for the contrast agent control computer 32 to take into account the investigation parameter. The detection of the investigation parameter is preferably effected before the generation of the at least one control parameter.

Furthermore, it is possible in the further step 201 of the method, in the generation of the control parameter, for the contrast agent control computer 32 to determine in addition a parameter for an expected complete exhaustion of the contrast agent that is still present within the contrast agent injector 27. The contrast agent parameter, determined by the contrast agent control computer, can form an input into the determination of the parameter for the expected complete exhaustion of the contrast agent that is still present, so that the parameter for the expected complete exhaustion of the contrast agent which is still present can be determined and/or ascertained as a function of the contrast agent parameter.

The parameter for the expected complete exhaustion of the contrast agent that is still present can then, in a further step 204 of the method, be presented using the presentation unit 34. In doing so it is possible to output and/or display the parameter for the expected complete exhaustion for medical operating staff by means of the user interface 24.

In step 201 of the method, for the generation of the control parameter, the contrast agent control computer 32 compares the parameter for the expected complete exhaustion of at least one of the contrast agents that is present within the contrast agent injector 27 with a threshold value. If the expected complete exhaustion of at least one of the contrast agents that is present within the contrast agent injector 27 falls below the threshold value, then a purchase order for a new quantity of the contrast agent is automatically generated by the contrast agent control computer 32. The contrast agent control computer 32 may also dispatch the purchase order. Alternatively, the purchase order can be presented to the medical operating staff, so that the dispatch of the purchase order is effected by the medical operating staff.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical imaging facility comprising:
a medical image signal detection unit having a patient receiving area therein configured to receive a patient or a medical imaging examination, said patient accommodation area being at least partially surrounded by said medical image signal detection unit; and
a contrast agent injector, configured to administer a contrast agent or a saline solution to the patient during said medical imaging examination, said contrast agent injector being built into said medical image signal detection unit.

2. A medical imaging facility as claimed in claim 1 wherein said medical imaging signal detection unit comprises a housing that shields said medical image signal detection unit and that at least partially surrounds said patient accommodation area, said housing comprising a contrast agent storage unit in which said contrast agent injector is situated.

3. A medical imaging facility as claimed in claim 2 wherein said housing comprises a housing shell on which said contrast agent storage unit is arranged.

4. A medical imaging facility as claimed in claim 2 wherein said housing comprises a housing shell having a recess therein, with said contrast agent storage unit being arranged at least partially within said recess.

5. A medical imaging facility as claimed in claim 2 wherein said contrast agent storage unit is situated outside of said patient accommodation area.

6. A medical imaging facility as claimed in claim 2 wherein said contrast agent storage unit is arranged on a front side of said housing.

7. A medical imaging facility as claimed in claim 1 comprising a contrast agent control unit used to control operation of said contrast agent injector, and that comprising a data link for exchanging data between said contrast agent control computer and said contrast agent injector.

8. A method for controlling a contrast agent injector that is built into a medical imaging facility, comprising:
determining at least one contrast agent parameter of said contrast agent injector with a contrast agent control computer in communication with said contrast agent injector;
in said contrast agent control computer, generating at least one control parameter, dependent on said at least one contrast agent parameter, for operating the contrast agent injector; and
controlling the contrast agent injector with said contrast agent computer using said at least one control parameter.

9. A method as claimed in claim 8 comprising before said contrast agent control computer generates said at least one control parameter, detecting an examination parameter for an impending medical imaging examination making use of said contrast agent injector, and generating said at least one control parameter in said contrast agent control computer dependent on said investigation parameter.

10. A method as claimed in claim 8 comprising detecting said at least one contrast agent parameter as a level parameter of at least one contrast agent in said contrast agent injector, or as a substance parameter for at least one contrast agent in said contrast agent injector.

11. A method as claimed in claim 8 comprising:
in said contrast agent control computer, using said at least one contrast agent parameter to determine at least one parameter relating to an expected complete exhaustion of said at least one contrast agent that is still present in said contrast agent injector; and
at a presentation unit in communication with said contrast agent computer, presenting said at least one parameter leading to the expected complete exhaustion of said at least one contrast agent.

12. A method as claimed in claim 11 comprising, in said contrast agent control computer, when said at least one contrast agent falls below a threshold value for said expected complete exhaustion of said at least one contrast agent, automatically generating a purchase order for a new quantity of said at least one contrast agent.

13. A method as claimed in claim 8 comprising:
in said contrast agent computer, detecting an event selected from the group consisting of an absence of said at least one contrast agent in said contrast agent injector, and a presence of a quantity of said at least one contrast agent when said contrast agent injector that is not sufficient to implement said medical imaging examination, having a warning message; and
emitting said warning message from said contrast agent control computer in a humanly perceptible form.

14. A non-transitory, computer-readable data storage medium encoded with programming instructions, said storage medium being loaded into a contrast agent control computer of a medical facility that has a contrast agent injector built therein, said programming instructions causing said contrast agent control computer to:
determine at least one contrast agent parameter for said contrast agent injector;
generate at least one contrast agent parameter dependent on said at least one control parameter; and
control said contrast agent injector using said at least one control parameter.
